(19) United States
(12) Patent Application Publication
(10) Pub. No.: US 2026/0123606 A1
(43) Pub. Date: May 7, 2026

(54) LOW-SALINITY TOLERANT, FAST-GROWING AND DISEASE RESISTANT OYSTER LINES

(71) Applicant: MORGAN STATE UNIVERSITY, Baltimore, MD (US)

(72) Inventor: Ming Liu, St. Leonard, MD (US)

(21) Appl. No.: 19/380,195

(22) Filed: Nov. 5, 2025

Related U.S. Application Data
(60) Provisional application No. 63/716,448, filed on Nov. 5, 2024.

(51) Int. Cl. A01K 61/54; C12Q 1/6888

(52) U.S. Cl. CPC A01K 61/54; C12Q 1/6888; C12Q 2600/124

(57) ABSTRACT

This invention relates to genetically improved Eastern oyster (Crassostrea virginica) lines developed by the Morgan State University Patuxent Environmental and Aquatic Research Laboratory. Derived from Maryland wild populations, these diploid, triploid, and tetraploid lines exhibit enhanced tolerance to low salinity, rapid growth, and disease resistance. Genetic improvement was achieved through multiple approaches including phenotype-based, genomic, and marker-assisted selection. Several diploid low-salinity-tolerant lines (LS2019, LS2025, LS H-GEBV) and a fast-growing line (FG H-GEBV) have been produced and are undergoing field evaluation, while MSX-resistant lines are under development through field challenge testing. Multi-trait genomic selection enables integration of growth, salinity tolerance, and disease resistance into single lines based on trait correlations and production needs. Triploid lines (3nD-20, 3nE-20, 3 nF-20, 3nW-25) generated by chemical induction are used to create tetraploids, ultimately producing superior commercial triploid seed. These distinct oyster lines improve aquaculture productivity and restoration success in low-salinity Chesapeake Bay environments.

### GOVERNMENT RIGHTS

This invention was made with government support under a Maryland Sea Grant award number NA180AR4170070 funded by the National Oceanic and Atmospheric Administration. The government has certain rights in the invention.

### FIELD OF THE INVENTION

This invention relates to marine aquaculture, and specifically to the selection and cultivation of low-salinity tolerant, fast-growing and disease resistant Maryland Oysters.

### BACKGROUND OF THE INVENTION

The development and propagation of oyster stock with desirable traits is crucial to the successful growth of the oyster aquaculture industry. Growth rate and survival are two important economic traits that oyster farmers actively pursue. In Maryland, oysters' growth and survival are challenged by low-salinity seawater and the prevalence of parasitic diseases. Selective breeding is an effective approach to achieve genetic improvement for desirable traits. Traditional selective breeding relies on phenotype and selection pressure, which requires continuous selection over many generations. Some traits are difficult to measure and require sacrificing the broodstock. In addition, selection pressure may be absent in some years, preventing continued selection. Both of these factors increase the difficulty and cost of selection and may reduce selection accuracy.

### SUMMARY OF THE INVENTION

The present invention relates to genetically improved Eastern oyster (Crassostrea virginica) lines developed by Morgan State University (MSU) Patuxent Environmental and Aquatic Research Laboratory (PEARL). These lines are derived from Maryland wild oyster populations and include diploid, triploid, and tetraploid forms that exhibit enhanced performance in growth rate, tolerance to low salinity seawaters, and resistance to diseases. The improved lines have been developed through traditional phenotype-based selection, genomic selection (GS), and/or marker-assisted selection (MAS) approaches. As a result, these lines are genetically distinct and unique from unselected wild populations and selected lines developed by other entities.

It is specifically noted that all oyster lines listed herein, as well as those that will be developed using the described methods at MSU PEARL, together with their progenies, derivatives, and hybrid offspring resulting from inbreeding or crossbreeding, are considered within the scope of the present invention.

### DETAILED DESCRIPTION OF THE INVENTION

### Diploid Oyster Lines.

### Low-Salinity Tolerant Diploid Oyster Lines.

### Method (1): Phenotype-Based Selection.

### Step (a): Low-Salinity Challenge Experiment to Select a Low-Salinity Tolerant Oysters.

An oyster line from the Patuxent wild population was produced in 2019 and labeled as PAX 2019. In the summer of 2021, 1,000 oysters randomly picked from PAX 2019 were used for a low-salinity challenge experiment at 2 ppt salinity. A total of 365 oysters survived when the challenge ended, labeled as LS 2019. In 2025, 20 large oysters from the survivors were used to produce the first generation (F1) LS line, labeled as LS 2025.

### Step (b): A Second Round of Selections.

In October 2025, when the LS 2025 seeds grow over 6 mm, five oyster bags will be deployed along with wild seed at the upper Patuxent to evaluate performance for two years. Survival will be checked every six months. An indoor low-salinity challenge will be performed when the seed is two years old to further confirm performance. Survivors from both field and indoor experiments will be preserved as more tolerant oysters after a second round of selection.

### Method (2): Genomic Selection (GS) or Marker-Assisted Selection (MAS).

Step (a): Construct GS Model and Identify Genetic Markers Associated with Low-Salinity Tolerance.

From phenotype-based selection steps, above, dead samples were collected and tissue preserved for DNA extraction. Survivors were biopsy-sampled after the experiment ended. Both dead and surviving samples were genotyped using a 66K SNP array. Phenotypes were recorded as dead=0 and alive=1. The estimated heritability for survival was 0.35.

After comparing several GS models—including genomic best linear unbiased prediction (GBLUP) and Bayesian Alphabet (Bayes A, Bayes B, Bayes Cπ, and Bayesian LASSO)—Bayes B was selected as the best-performing model, achieving the highest prediction accuracy (0.764) based on five-fold cross-validation. Additionally, Genome-wide Association analysis identified 30 significant SNP outliers, suggesting that selection could be made more cost-effective by enabling marker-assisted selection.

Step (b): Using the GS Model to Select Low-Salinity-Tolerant Broodstock from Patuxent Wild Population.

A total of 300 wild oyster broodstock were collected from the Patuxent River population, biopsy-sampled, and genotyped using the 66K SNP array in Spring 2024. The GS model for low-salinity tolerance was applied to calculate the genomic estimated breeding value (GEBV) for each broodstock. The top 30 oysters with the highest GEBVs were designated as tolerant stock (LS H-GEBV), and the bottom 30 as intolerant stock (LS L-GEBV). Offspring from both LS H-GEBV and LS L-GEBV groups were produced in Summer 2025. These will be deployed at extreme low-salinity sites (<5 ppt) and tested in indoor low-salinity challenge experiments to evaluate the GS model's predictive effectiveness.

### Fast-Growing Diploid Oyster Lines.

### Step (a): Construct GS Model for Fast-Growth Trait.

In 2021, growth metrics (shell height, shell length, shell width, and total weight) were recorded for 1,000 oysters randomly selected from PAX 2019. Among these, 768 oysters were genotyped for the low-salinity challenge experiment, allowing linkage between genotype and growth traits. The heritabilities of shell height (SH) and total weight (TW) were 0.21 and 0.11, respectively. The average accuracy of single-trait models was 0.55 for SH and 0.69 for TW using GBLUP. Multi-trait analysis revealed significant phenotypic and genetic correlations between SH and TW, and the average accuracy for SH increased to 0.94 when TW was included.

### Step (b): Using GS Model to Select Fast-Growing Broodstock.

A total of 300 wild oyster broodstock were collected from the Patuxent River population, biopsy-sampled, and genotyped using the 66K SNP array in Spring 2024. The multi-trait GS model for growth traits was applied to calculate the GEBV for each broodstock. The top 30 oysters were preserved as fast-growing stock (FG H-GEBV), and the bottom 30 as control stock (FG L-GEBV).

### Low-Salinity and Fast-Growing Diploid Oyster Lines.

Low-salinity tolerance and growth traits were found to be uncorrelated, indicating that selection for both traits is feasible. The two traits can be combined by calculating an aggregate GEBV from the existing GS models for low-salinity tolerance and growth. The equation is: I*=p₁GEBV₁*+p₂GEBV₂*, where p₁ and p₂ are the weighting indexes of the traits, I* is the aggregate GEBV, and GEBV* represents the normalized breeding values. The normalization equation is: GEBV*=(GEBV−Y̅GEBV)/σGEBV, where GEBV is the original breeding value of an individual, Y̅GEBV is the mean GEBV of the population, and σGEBV is the standard deviation of GEBVs in the population.

### Disease-Resistant Diploid Oyster Lines.

### Method (1): Phenotype-Based Selection.

Step (a): Deploy LS 2025 and a Control Line (Derived from Wild Population) at High MSX Disease-Prevalent Sites to Select MSX-Resistant Oysters.

In October 2025, when the LS 2025 seeds and its control wild line grow over 6 mm, ten bags of each will be deployed at two high-salinity, MSX disease-prevalent sites for two years. Survival and infection status will be checked every six months. Survivors exhibiting resistance will form the foundation population for further selection.

### Method (2): Genomic Selection or Marker-Assisted Selection.

Step (a): Construct GS Model and/or Identify Genetic Markers Associated with Disease Resistance.

Two thousand labeled one-year-old oysters from LS 2025 (1,000) and its control line (1,000) will be genotyped using the 66K SNP array and before deploying at a high-disease-mortality site. After mortality occurs, the status of each oyster can be determined from its label, survival or dead. The GS model will be constructed based on the phenotype and genotype, similar to the low-salinity trait. Heritability and GWAS will also be performed to identify significant SNPs.

### Step (b): Utilize GS or MAS to Predict Disease-Resistant Lines.

If disease resistance shows high heritability and significant markers are identified, MAS will be used to develop MSX-resistant oyster lines. If the heritability is low, GS will be used. The first batch of disease-resistant broodstock will be selected from the 300 known-genotype oysters.

### Low-Salinity and Disease-Resistant or Combined Multi-Trait Diploid Oyster Lines.

As with previous analyses, phenotypic and genetic correlations between traits will be examined. If no negative correlations are detected, two or more traits (low-salinity tolerance, growth, and disease resistance) can be combined using the aggregate GEBV approach described above.

### Triploid Oyster Lines.

Triploid oyster lines were developed through chemical (6-dimethylaminopurine, cytochalasin B) induction or mass selection from wild populations or superior diploids developed at PEARL. These serve as foundational material for creating tetraploid stock.

In June 2020, 50 oysters were collected from the Patuxent wild population. Based on shell height (SH) measurements, the top 12 and bottom 16 oysters were selected and divided into two groups. After sex determination and gamete grading, five females and one male from the top group were used to create the triploid line 3nD-20 by inhibiting Polar Body II. Eight females and four males from the lower group were used to fertilize eggs divided equally into two batches: one treated to create 3nE-20 (inhibiting Polar Body I) and the other to create 3 nF-20 (inhibiting Polar Body II).

All three triploid lines were deployed at the PEARL pier. Mortality and growth metrics were recorded at 11, 17, and 28 months. Line 3nD-20 exhibited superior growth compared to other lines.

In Summer 2025, to maintain adequate triploid broodstock for tetraploid development, a new triploid line, 3nW-25, was bred from the Patuxent wild population using CB chemical induction.

### Tetraploid Oyster Stock.

Tetraploid oysters will be developed using above triploid and diploid lines. The standard method involves inhibiting the release of Polar Body I in fertilized eggs from triploid females crossed with diploid males using CB treatment.

### Commercial Triploid Seed.

When tetraploid broodstock are available, commercial triploid seed for aquaculture will be produced by crossing tetraploids with superior diploids developed by the inventors.

In summary, the triploid and diploid oyster lines described herein, as well as populations or lines developed at MSU PEARL using the MAS or GS methods described herein are considered to fall within the scope of the inventions described herein.

Notwithstanding the specific embodiments, features, elements, combinations and sub-combinations disclosed herein, it is expressly considered and here disclosed that every single element, every single feature, and every combination and sub-combination thereof disclosed herein may be combined with every other element, feature, combination and sub-combination disclosed herein.

It will be appreciated by those skilled in the art that changes could be made to the embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. An Diploid Eastern oyster (Crassostrea virginica) line developed from a Maryland wild population at the Morgan State University Patuxent Environmental and Aquatic Research Laboratory (MSU PEARL), the line being genetically distinct from the wild population and characterized by enhanced performance in at least one of:
(a) tolerance to low salinity;
(b) accelerated somatic growth; and
(c) resistance to disease,
wherein the line is produced by (i) phenotype-based selection under a defined challenge condition and/or (ii) genomic selection (GS) and/or marker-assisted selection (MAS).

2. The oyster line of claim 1, wherein the line is a diploid low-salinity-tolerant line comprises at least one of LS 2019, LS 2025, or LS H-GEBV, and their progenies.

3. The oyster line of claim 1, wherein the line is a diploid low-salinity-tolerant line that is produced using the genomic selection model or the 30 significant SNP markers associated with low-salinity survival.

4. The oyster line of claim 1, wherein the line is a fast-growing diploid line selected using a multi-traits (shell height and total weight) genomic selection model.

5. The oyster line of claim 11, wherein the line is a fast-growing diploid line comprises FG H-GEBV or its progenies.

6. The oyster line of claim 1, wherein low-salinity tolerance and fast growth are genetically uncorrelated, and the two traits are combined in a single individual by computing an aggregate genomic selection index.

7. The oyster line of claim 1, wherein the enhanced trait comprises resistance to MSX disease, the resistance being developed by deploying selected and control lines at two high-salinity, MSX-prevalent field sites for at least two years and retaining survivors as disease-resistant broodstock.

8. The oyster line of claim 1, wherein the line is a diploid MSX resistant line that is produced using the genomic selection model or SNP markers associated with disease-resistance.

9. The oyster line of claim 1, wherein the line simultaneously possesses (i) low-salinity tolerance and (ii) MSX resistance, the two traits having been tested for adverse genetic correlation and, when no negative correlation is detected, integrated into a single selection index as in claim 6.

10. The oyster line of claim 1, wherein the line further possesses (iii) fast growth, and low-salinity tolerance, disease resistance, and fast growth are combined in a three-trait aggregate GEBV.

11. A triploid Eastern oyster line produced from Patuxent River wild broodstock or from a PEARL-developed superior diploid line, the triploid line being generated by chemical inhibition of polar body I or polar body II during early embryogenesis.

12. The triploid line of claim 11, wherein the triploid is one of 3nD-20, 3nE-20, 3 nF-20, or 3nW-25, and their progenies.

13. A tetraploid eastern oyster stock produced by crossing triploid females from any of the lines of claims 11 and 12 with a diploid male from any of the lines of claims 1-10, and inhibiting polar body I release using cytochalasin B.

14. Commercial triploid seed comprising crossing the tetraploid stock of claim 13 with any of the improved diploid lines of claims 1-10, thereby generating triploid progeny combining low-salinity tolerance, fast growth, and/or disease resistance.

15. The oyster line, stock, or progeny of any of claims 1-24, wherein the line is intended for use in oyster aquaculture, spat-on-shell production, or estuarine restoration in low-salinity regions of the Chesapeake Bay and adjacent Atlantic estuaries.
